(19) United States
(12) Patent Application Publication
(10) Pub. No.: US 2020/0078562 A1
(43) Pub. Date: Mar. 12, 2020

(54) SECUREMENT DEVICES FOR MEDICAL ARTICLES

(71) Applicant: CleanSite Medical, Inc., San Diego, CA (US)

(72) Inventor: Daniel M. CHAMBERS, Solana Beach, CA (US)

(73) Assignee: CleanSite Medical, Inc., San Diego, CA (US)

(21) Appl. No.: 16/543,545

(22) Filed: Aug. 17, 2019

Related U.S. Application Data
(60) Provisional application No. 62/719,198, filed on Aug. 17, 2018.

(51) Int. Cl. A61M 25/02

(52) U.S. Cl. CPC A61M 25/02; A61M 2025/0266

(57) ABSTRACT

Medical article securement devices are described. These devices retain a medical article, for example, a catheter hub or other luer access device, in position on a patient's body and reduce or inhibit the article's longitudinal or other movement.

### RELATED APPLICATION(S)

This application claims the benefit of and priority to U.S. provisional patent application No. 62/719,198, filed on 17 Aug. 2018, the contents of which is hereby incorporated by reference in its entirety for any and all purposes.

### TECHNICAL FIELD OF THE INVENTION

This invention is directed to cleansing devices for cleansing and capping medical devices, particularly luer access devices such as needleless, valved connectors, and methods for using such articles.

### BACKGROUND OF THE INVENTION

### 1. Introduction

The following description includes information that may be useful in understanding the present invention. It is not an admission that any such information is prior art, or relevant, to the presently claimed inventions, or that any publication specifically or implicitly referenced is prior art.

### 2. Background

Modern medical practice frequently involves using catheters to introduce fluids and medications directly into a patient's vasculature. Examples include peripheral intravenous catheters (PIVCs), peripherally inserted central catheters (PICCs), and central venous catheters (CVCs). Often, it is necessary to maintain such catheterization over an extended period during a patient's treatment, particularly when the patient is hospitalized. In order to keep a catheter (or other medical line) properly positioned for as long as needed, the catheter (or other medical line) is typically secured to the patient. Historically, securement involved taping the catheter or medical line to the patient.

Securing catheters to a patient using tape, however, has several drawbacks. First, the use of tape to secure a catheter can retain dirt or other contaminants at or near the catheter insertion site (the opening in the patient's skin created by inserting the catheter), potentially leading to infection or other complications. Indeed, numerous clinical studies have implicated improperly secured catheters in a wide range of complications, including catheter-related blood stream infections (CRBSI), which lead to nearly 90,000 deaths annually in the U.S. alone. For example, tape-based securement may not adequately control catheter “pistoning” (i.e., catheter movement further into or being partially withdrawn from the insertion site), pitch, roll, and/or yaw movement, etc. Second, failing to limit catheter movement can contribute to motion-related complications such as bruising, phlebitis, extravasation, infiltration, and catheter migration, which can lead to catheter dislodgement or disconnection. Third, tape removal can itself cause undesired catheter movement. Fourth, tape must periodically be changed, often daily. The frequent removal and reapplication of adhesive tape can irritate a patient's skin, as well as lead to the build up of adhesive residue on the outer surface of the catheter (or other medical line). Such adhesive residue not only makes the catheter (or other medical line) stickier and more difficult for healthcare providers to handle (as being “sticky” causes the article to “stick to” gloves, etc.), it can also result in contaminants (including pathogens) adhering to the catheter, increasing the likelihood of infection, either at the skin surface or internally. Fifth, tape securement can allow medical lines attached to a catheter to flex or kink, which can lead to clinical complications.

As a result of these drawbacks, other approaches to securing catheters and medical lines have been sought. One such solution is reported in U.S. Pat. Nos. 7,014,627 and 7,799,001. However, such devices themselves have many drawbacks, including high unit cost, difficulty in using such devices, and the need for hospitals to stock large inventories because individual devices are not adaptable to fit the wide range of different catheters and medical articles routinely in use today.

Given the shortcomings of currently available catheter (and medical line) securement technologies, a significant unmet need still exists for inexpensive, low-profile, easy-to-use medical article securement devices.

### 3. Definitions

Before describing the instant invention in detail, several terms used in the context of the present invention will be defined. In addition to these terms, others are defined elsewhere in the specification, as necessary. Unless otherwise expressly defined herein, terms of art used in this specification will have their art-recognized meanings.

The term “adaptive” refers to a device's or structure's ability to adapt and substantially conform to the exterior shape and/or contours of another article, including a plurality of different articles having different exterior dimensions and shapes (e.g., catheter hubs having different outer dimensions). Features that provide adaptive functionality include hinges (including combinations of forward and reverse hinges), crushable tabs, fins, and the like.

A “luer access device”, or “LAV”, refers to a medical article having at least one luer fitting and which provides access to a patient's vasculature. Such devices include PICCs, PIVCs, CVCs, and the like.

A “medical article” refers to catheters, catheter hubs, medical line connector fittings, luer access devices, medical lines (e.g., tubing intended to deliver fluids to a patient), and the like.

A “patentable” composition, process, machine, or article of manufacture according to the invention means that the subject matter at issue satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically excludes the unpatentable embodiment(s). Also, the claims appended hereto are to be interpreted both to provide the broadest reasonable scope, as well as to preserve their validity. Furthermore, if one or more of the statutory requirements for patentability are amended or if the standards change for assessing whether a particular statutory requirement for patentability is satisfied from the time this application is filed or issues as a patent to a time the validity of one or more of the appended claims is questioned, the claims are to be interpreted in a way that (1) preserves their validity and (2) provides the broadest reasonable interpretation under the circumstances.

A “plurality” means more than one. The term “resilient” refers to a structure or component able to recoil or spring back into its original shape after bending, compression, flexion, or stretching.

### BRIEF DESCRIPTION OF THE DRAWINGS

FIGS. 1-11 depict different representative, non-limiting examples of the invention. In the drawings, like numbers in different drawings indicate the like elements.

FIGS. 12a-12b depict different representative, non-limiting examples of the invention. In the drawings, like numbers in different drawings indicate the like elements.

### THE INVENTION

The following description and the accompanying drawings, which describe and show the preferred embodiments, demonstrate several possible representative configurations that a medical article securement device of the invention can take to include various aspects and features the invention. These illustrations are not to scale and are not intended to limit the invention to the specified embodiments or usage. Those skilled in the art will recognize that the described aspects and features of the invention are not limited to any particular embodiment of a securement device, and securement devices according to the invention can readily be designed for use with a variety of medical articles.

The devices and methods of the present invention have several features, no single one of which is solely responsible for its desirable attributes. Without limiting the scope of this invention as expressed by the appended claims, its more prominent features will now be summarized.

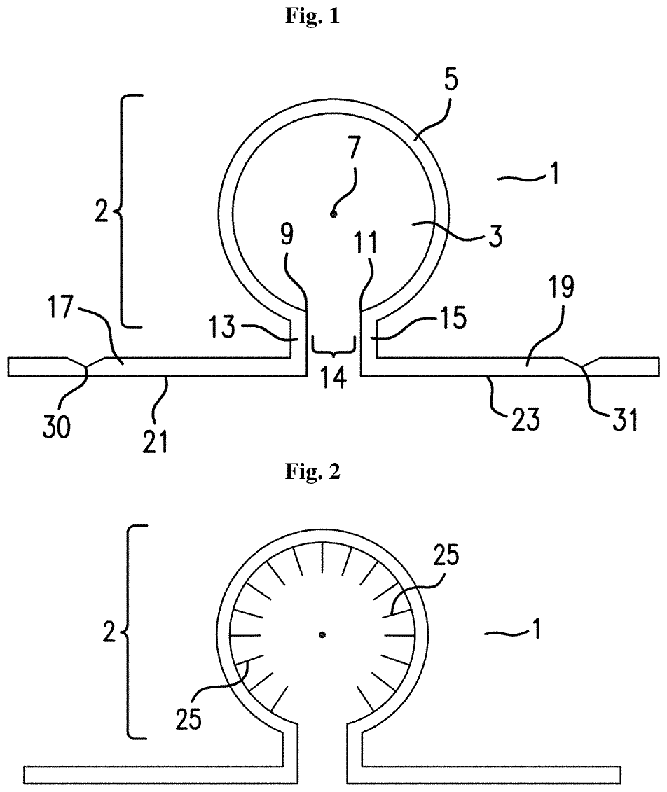

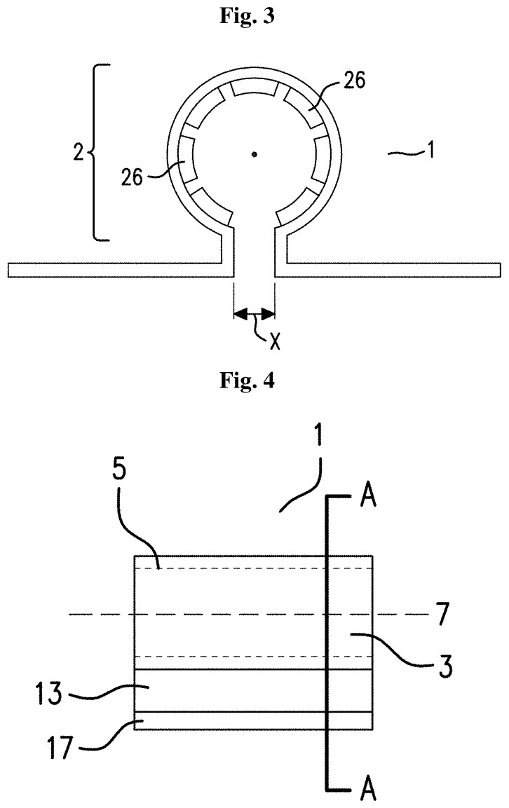

One object of the invention concerns resilient medical article securement devices (1). Such devices comprise a catheter (or catheter hub or other luer access device) engaging member (2) that includes a channel (3) formed by a resilient channel wall (5) that bounds a central axis (7). The resilient channel wall (5) in cross section (see, e.g., FIGS. 1-3) typically forms a downwardly extending arc, for example, an open circle or portion thereof, that extends around the central axis (7) more than 180 degrees and has first and second ends or termini (9, 11) that can move away from and toward one another, as when grasped by a nurse to use the resilient securement device to secure a medical article to a patient. As will be appreciated, the engaging member is made of a resilient material that allows the first and second ends or termini (9, 11) to return to their respective positions after a force applied to open the device (e.g., by grasping) is relieved. In some embodiments, there is a gap or opening (14) between the first and second ends or termini (9, 11) when the device is in a neural, unstressed position. In other embodiments, there is little or no appreciable gap (14) when the device is in a neural, unstressed position.

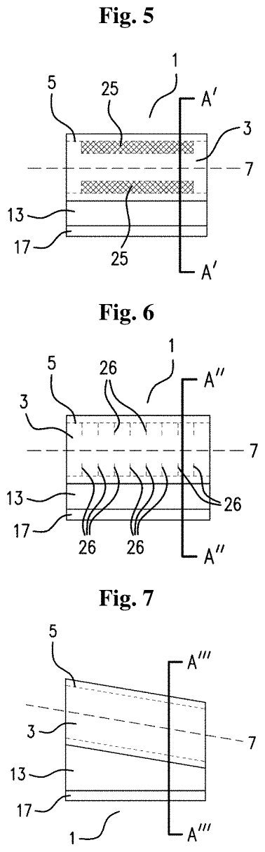

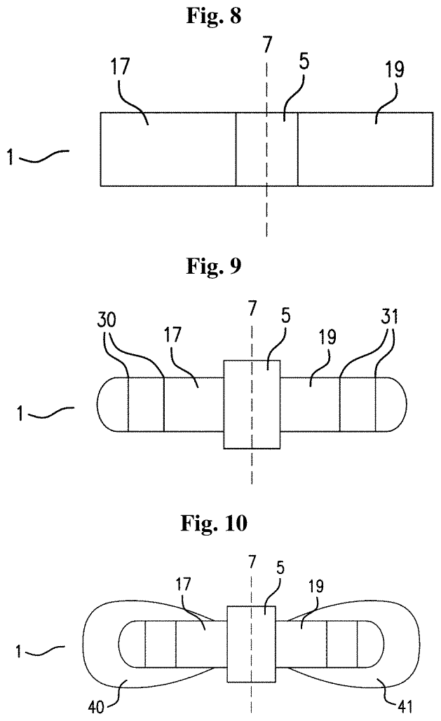

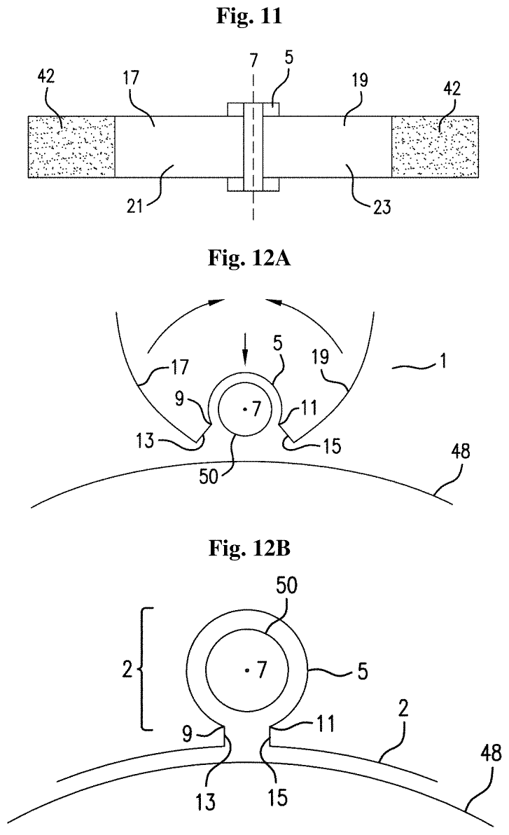

The devices of the invention also include first and second support struts (13, 15) that support and elevate the catheter-engaging member (2) above resilient securement members (17, 19) used to adhere the device (1) to a patient's skin. See, e.g., FIGS. 1 and 4-7. Each strut (13, 15) has an upper portion and a lower portion. The upper portion of the first strut (13) is connected to the first end (7) of the resilient channel wall (5) and the lower portion of the first strut (13) is connected to a first resilient securement member (17) that extends away from the second end (11) of the resilient channel wall (5) and whose lower surface comprises an adhesive (42) or adhering structure over at least portion thereof. The second support strut (15) also has an upper portion and a lower portion. The second strut's upper portion is connected to the second end (11) of the resilient channel wall (5) and its lower portion is connected to a second resilient securement member (19) that extends away from the first end (9) of the channel wall (5) and whose lower surface comprises an adhesive (42) or adhering structure over at least portion thereof. The first and second resilient securement members (17, 19) together are configured to provide, through adhesive portions (42) or adhering structures disposed on their respective lower surfaces (21, 23), adherence of the medical article to a patient's skin.

In some embodiments of this aspect, a middle portion is disposed between the upper and lower portions of the first and second support struts (13, 15). The profile of support struts can be rectangular or tapered, with a tapered profile (i.e., when viewed from the side) being preferred. In preferred embodiments, the struts elevate the channel portion (3) of the device such that a when a medical article is secured in the device, the medical article is lifted above the patient's skin so as to prevent contact between the secured medical article and the patient over the length of the medical article disposed in the channel of the device. For example, when the device of the invention is used to secure a peripheral IV catheter to a patient's skin, the device elevates the hub portion of the catheter above the patient's skin and, in preferred embodiments, angles the catheter hub such that the catheter portion that protrudes therefrom and is inserted into the patient's vein is maintained at a desired angle (less than 45, preferably between about 5-30, particularly about 7 to about 22, with 7 being particularly preferred; see, e.g., U.S. Pat. No. 6,673,046), which prevents catheter kinking, undue stress on the patient's skin at the catheter insertion site, etc.

In some embodiments, at least one of the first and/or second resilient securement members (17, 19) of the medical article securement devices (1) further comprise at least one hinge (30, 31) to promote conformance to patient anatomy.

In some embodiments, the medical article securement devices further comprises at least one engaging element, e.g., a slot or other opening in the channel wall, configured to engage a retention element (e.g., a push plate) disposed on the surface of the medical article so as to reduce or inhibit movement, optionally longitudinal and/or pitch and/or roll and/or yaw movement, of the medical article when secured by the securement device of the invention.

In some embodiments, the medical article securement devices are adapted to engage a catheter hub or medical connector fitting, for example, a luer access device or luer activated valve.

In some preferred embodiments of the invention, the medical article securement devices are configured to adapt to a plurality of medical devices of different sizes. Such adaptability can be provided by one or more adaptive elements, for example, a plurality of crushable structures (e.g., crushable fins (25), tabs (26), fingers, and the like) disposed on the inner surface of the resilient channel wall (5).

In some embodiments, the channel (3) of a medical article securement device of the invention has a tapered shape configured to complement a cooperative tapered shape of the portion of the medical article to be disposed in the central channel.

In preferred embodiments of the invention, the first and second securement members (17, 19) of the medical article securement devices comprise adhesive portions (42) or adhering structures disposed on their respective lower surfaces. Preferably, in such devices the adhesive portions or adhering structures disposed on the first and second securement members each further comprises a removable layer, for example a release liner, that a nurse of other medical professional will remove just before adhering the device to a patient in order to secure the medical article associated therewith.

A related aspect of the invention concerns medical article securement system, which comprise a medical article securement devices of the invention operably associated with a medical article (e.g., a luer activated needleless medical valve), which system can be used to securely attach the medical article to a patient.

Another aspect relates to methods of uniting a medical article with an adaptive medical article securement device of the invention. Such methods involve placing a medical article securement device according to invention in securing relation (i.e., operable association) with a medical article so that the channel of the device engages and retains the medical article, thereby uniting the medical article and securement device.

As will be appreciated, the medical article securement devices of the invention can be used to secure a medical article to a patient by adhering the adhesive portions of the device to a patient's skin. In practice, this will typically involve a nurse apply force to the resilient securement members (17,19), causing them to bend upward, which also causes the first and second ends (9, 11) and first and second struts (13, 15) to move apart, thereby widening the space (X) between the first and second struts to facilitate its lowering into position to secure a medical article (50) to a patient's skin (48).

Other aspects, embodiments, features, and advantages of the invention will be apparent from the drawings, the disclosure below, and the appended claims.

To assist in the description of the invention, the following coordinate terms are used. A “central” axis” refers an axis locate at the center of the channel in the catheter engaging member of a resilient securement device of the invention. A “longitudinal axis” is generally parallel to a portion of the catheter hub, the connector fitting, or other medical article retained by the an adaptive medical article securement device of the invention, as well as parallel to the axis of the central channel of the adaptive retainer, through which the medical article extends. A “lateral axis” is normal or perpendicular to the longitudinal or central axis, as the context requires or as indicated. In addition, “longitudinal direction” refers to a direction substantially parallel to the longitudinal axis and “lateral direction” refers to a direction substantially parallel to the lateral axis. The term “axial” refers to the axis of the central channel or connector fitting, and therefore is substantially synonymous with the term “longitudinal” as used herein. Also, the terms “proximal” and “distal”, are used consistently, and in context refer to near and far, respectively. The terms “upper,” “lower,” “top,” “bottom,” “underside,” “upperside”, and the like, are used in reference to the orientation of the particular embodiment.

The preferred embodiments of the present invention advantageously provide medical article securement devices for securing a medical article to a patient. The medical article preferably has an elongated body that cooperates with the adaptive retainer to arrest movement of the medical article in longitudinal, lateral, and other directions when placed within the channel of the medical article securement device.

This invention concerns a new generation of patentable devices for securing medical articles (e.g., medical lines, needles, catheters, etc.) to a patient's skin. In preferred embodiment, the securement devices of the invention can be readily adapted to fit a wide range of medical article types, combinations, and dimensions for easy, rapid, and secure attachment to a patient's skin. The devices of the invention are also inexpensive and efficient to manufacture. Finally, the resilient nature of the channel wall and other portions of the securement device makes them easy to use to secure a medical article to a patient's skin.

The devices of the invention further include anchor regions (40, 41) disposed at opposite ends of the device, typically on portion of the lower surfaces of the first and second resilient securement members. When operably associated with a medical article to secure the latter to a patient, the securement device holds the retained portion of the medical article away from the patient's skin when the retained portion is positioned within the channel of the securement device. This avoids chaffing or otherwise irritating the patient's skin. In some preferred embodiments, a securement devices of the invention may also include a removal tab disposed at one or both ends of the anchor regions (or anchor pad) to allow for the securement device's easy removal from a patient. Removal tabs typically do not include an adhesive.

A securement device of the invention can have other constructions to minimize contact between the patient's skin and the securement device, as well as between the patient and the retained portion of the medical article. For example, anchor pads, if provided, can be thicker, in which case the mounting wings can be located higher on the securement device.

In embodiments have at least two anchor regions, the anchor regions are preferably disposed opposite each other at or near the distal ends of the first and second resilient securement members. The anchor regions also have an adhesive located on the surface intended for skin contact. The adhesive should be suitable for securing the article to a patient and for prolonged skin contact (i.e., for more than one hour, up to and including 1, 3, 5, 7, or more days).

In order to protect the adhesive characteristics of the anchor regions, the securement devices of the invention are manufactured to include a removable layer (e.g., a release liner) attached to those surfaces to which an adhesive layer has been applied. Just prior to use the removable layer(s) can be separated from (e.g., by peeling) the rest of the device, after which the removable layer(s) can be disposed of.

All of the devices, articles, systems, and methods described and claimed herein can be made and executed without undue experimentation in light of the specification and drawings. While the invention has been described in terms of preferred embodiments, it will be apparent to those of ordinary skill in the art that variations may be applied to the devices, articles, systems, and methods without departing from the spirit and scope of the invention. All such variations and equivalents apparent to those skilled in the art, whether now existing or later developed, are deemed to be within the spirit and scope of the invention as defined by the appended claims.

All patents, patent applications, and publications mentioned in the specification are indicative of the levels of those of ordinary skill in the art to which the invention pertains. All patents, patent applications, and publications are herein incorporated by reference in their entirety for all purposes and to the same extent as if each individual publication was specifically and individually indicated to be incorporated by reference in its entirety for any and all purposes.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A resilient medical article securement device, comprising:
(a) a catheter engaging member that comprises a channel formed by a resilient channel wall that bounds a central axis, wherein the resilient channel wall in cross section forms a downwardly extending arc, preferably an open circle, that extends around the central axis more than 180 degrees and has first and second ends that can move away from and toward one another;
(b) a first support strut having an upper portion and a lower portion, the upper portion being connected to the first end of the resilient channel wall and the lower portion being connected to a first resilient securement member that extends away from the second end of the channel wall and whose lower surface comprises an adhesive or adhering structure over at least portion thereof; and
(c) a second support strut having an upper portion and a lower portion, the upper portion being connected to the second end of the resilient channel wall and the lower portion being connected to a second resilient securement member that extends away from the first end of the channel wall and whose lower surface comprises an adhesive or adhering structure over at least portion thereof, wherein the first and second resilient securement members are together configured to provide, through adhesive portions or adhering structures disposed on their respective lower surfaces, adherence of the medical article to a patient's skin.

2. A device according to claim 1 wherein the first and second support struts have a middle portion disposed between the upper and lower portions that has a profile, preferably a tapered profile, and spaces the upper and lower portions of each strut a distance sufficient to elevate a medical article secured by the device a distance above the patient's skin that prevents contact between the secured medical article and the patient over the length of the medical article disposed in the channel of the device.

3. A device according to claim 1 wherein at least one of the first and/or second resilient securement members further comprise at least one hinge to promote conformance to patient anatomy.

4. A device according to claim 1 that further comprises at least one engaging element configured to engage a retention element disposed on the surface of the medical article so as to reduce or inhibit movement, optionally longitudinal and/or pitch and/or roll and/or yaw movement, of the medical article.

5. A device according to claim 1 wherein the channel is adapted to engage a catheter hub or connector fitting, optionally a luer access device or luer activated valve.

6. A device according to claim 1 wherein the channel is configured to adapt to a plurality of medical devices of different sizes, wherein the inner surface of the resilient channel wall includes at least one adaptive element, optionally a plurality of crushable structures.

7. A device according to claim 1 wherein the channel has a tapered shape configured to complement a cooperative tapered shape of the portion of the medical article to be disposed in the central channel.

8. A device according to claim 1 wherein the first and second resilient securement members further comprise adhesive portions or adhering structures disposed on their respective lower surfaces.

9. A device according to claim 8 wherein the adhesive portions or adhering structures disposed on the first and second resilient securement members further each comprises a removable layer.

10. A medical article securement system, comprising a device according to claim 1 securing a resilient medical article to a patient.

11. A method of uniting a medical article with a resilient medical article securement device, comprising placing a device according to claim 1 in securing relation with a medical article so that the channel of the device retainingly engages the medical article, thereby uniting the medical article and the resilient medical article securement device.

12. A method of securing a medical article to a patient, comprising uniting a medical article with a device according to claim 1 and adhering the adhesive portions of the device to a patient's skin.
